(19) United States
(12) Patent Application Publication
(10) Pub. No.: US 2023/0390142 A1
(43) Pub. Date: Dec. 7, 2023

(54) SHOULDER BELT MASSAGE MACHINE CORE

(71) Applicant: Xiamen Emoka Health Science & Technology Co., Ltd., Xiamen (CN)

(72) Inventor: Guanghua ZHOU, Xiamen (CN)

(21) Appl. No.: 18/451,752

(22) Filed: Aug. 17, 2023

(30) Foreign Application Priority Data
Jul. 27, 2023 (CN) 202321994285.6

(51) Int. Cl. A61H 7/00; A61F 7/00

(52) U.S. Cl. CPC A61H 7/007; A61F 7/007

(57) ABSTRACT

The present disclosure discloses a shoulder belt massage machine core, including: a housing, a battery, a brushless motor, a circuit board, a gear transmission group, a long rotating shaft, eccentric wheels, yaw wheels, yaw wheel covers, ejector rods, yaw arms and massaging assemblies; The overall structure of the present disclosure is more compact and can be manufactured at a lower cost, so that the competitiveness of the market can be improved.

### CROSS-REFERENCE TO RELATED APPLICATIONS

This application claims the benefit of priority from Chinese Patent Application No. 202321994285.6, filed on Jul. 27, 2023. The content of the aforementioned application, including any intervening amendments thereto, is incorporated herein by reference in its entirety.

### TECHNICAL FIELD

The present disclosure relates to the technical field of massaging machines, and in particular to a shoulder belt massage machine core.

### BACKGROUND

In modern social life, people work for a long time in the office, study for a long time in the classroom, and play with computers and mobile phones for a long time in the dormitory or at home, which inevitably causes many people to suffer from cervical spondylosis and scapulohumeral periarthritis day after day. Shoulder belt massagers for shoulder and neck massage came into being. The shoulder belt massager is mainly used to massage the shoulder and neck positions of users. When in use, mechanical kinetic energy is provided for a massage assembly through a driving device, so that the massage assembly can relax muscles and activate collaterals of users by regular rotation, swing, vibration and beating, improve blood circulation of shoulders and neck, and relieve symptoms such as rigidity and pain of shoulder and neck muscles. The existing shoulder and neck massager with a kneading function is usually provided with a finger massage assembly and a kneading massage assembly for realizing finger massage and kneading massage, and the finger massage assembly and kneading massage assembly are respectively driven by corresponding driving mechanisms, so that the overall volume of the massager is larger, the production cost is also improved, and the market competitiveness is not strong.

In view of the above, the present designers have developed the present disclosure by intensively conceiving the disadvantages and inconveniences caused by the unperfect design of the conventional shoulder belt massage machine core, and actively researching and improving trials.

### SUMMARY

The disclosure aims at overcoming the shortcomings of the prior art and providing a shoulder belt massage machine core with a compact structure and a lower cost.

In order to achieve the above objective, the solution of the present disclosure is:
a shoulder belt massage machine core includes a housing, a battery, a brushless motor, a circuit board, a gear transmission group, a long rotating shaft, eccentric wheels, yaw wheels, yaw wheel covers, ejector rods, yaw arms and massaging assemblies; the battery, the brushless motor, the gear transmission group and the long rotating shaft are arranged inside the housing, the battery is connected with the circuit board, the brushless motor is connected with the circuit board, the gear transmission group is connected with an output shaft of the brushless motor, the long rotating shaft is connected with the gear transmission group, the two eccentric wheels are fixedly arranged on both sides of the long rotating shaft corresponding to the gear transmission group, the ejector rod is arranged on the eccentric wheel in a sleeving mode, one end of the ejector rod connected with the eccentric wheel is limited on the housing by an ejector rod pull tab, the yaw wheels are fixedly arranged outside the long rotating shaft corresponding to the two eccentric wheels, a side of the yaw wheel facing away from the eccentric wheel is provided with a yaw shaft, one end of the yaw arm is provided with a yaw hole corresponding to the yaw shaft, the yaw hole is arranged on the yaw shaft in a sleeving mode, the yaw shaft of the yaw wheel and one end connected with the yaw arm form a yaw slope, the bottom of one end of the yaw arm connected with the long rotation shaft is provided with a guide block, the guide block is movably disposed in a guide groove of the housing, the yaw hole is disposed corresponding to the yaw slope, the other end of the yaw arm is provided with a massage seat, the massage assembly is mounted on the massage seat, the yaw wheel cover is arranged on the long rotating shaft in a sleeving mode and fixed with an outer end surface of the yaw shaft of the yaw wheel.

Further, the gear transmission group includes a double gear having a helical gear and a spur gear and a driven gear fixed on the long rotating shaft, the helical gear and the spur gear of the double gear are fixed together through a gear rotating shaft, the double gear is mounted on a gear box, the gear rotating shaft is parallel with the long rotating shaft, two ends of the gear rotating shaft are disposed on the gear box; the brushless motor is provided with a worm, the worm is perpendicular to the long rotating shaft and is arranged below the long rotating shaft, one end of the worm is connected with the brushless motor, the other end is sleeved with a fixing sleeve, and the fixing sleeve is fixed on the gear box; the helical gear of the double gear meshes with the worm and the spur gear meshes with the driven gear.

Further, the gear transmission group is mounted on a gear box which is detachably mounted on the housing 1.

Further, the long rotating shaft is further provided with bearings on both sides of the driven gear, the bearing being provided between the eccentric wheel and the driven gear, wherein a Hall plate is further provided between one eccentric wheel and the bearing.

Further, the shoulder belt massage machine core of the disclosure further includes a heating sheet connected with the circuit board, the heating sheet having a temperature control sensor built therein, and the heating sheet being disposed on an inner top surface of the housing.

Further, the housing includes a lower housing and an upper cover, the center of the lower housing is provided with a chamber for mounting the gear box and the brushless motor, and the upper cover covers the chamber, and the heating sheet is fixed in the upper cover.

Further, the massage assembly includes a first massage head and a duplex massage head, the massage seat of the yaw arm includes a first massage seat extending from the yaw arm to one side of a free end of the ejector rod and a second massage seat extending from the other side, the first massage head is mounted on the first massage seat, and the duplex massage head is mounted on the second massage seat.

Further, the second massage seat is an elastic block, and the duplex massage head has a mounting opening corresponding to the elastic block, the mounting opening is arranged on the elastic block in a sleeving mode.

Further, the eccentric wheel, the ejector rod and the yaw arm are respectively provided with a plurality of oil storage grooves, the oil storage grooves of the eccentric wheel are provided at one end surface of the eccentric wheel connected with the ejector rod, two end surfaces of the ejector rod connected with the eccentric wheel and the yaw wheel are respectively provided with the oil storage grooves, and two end surfaces of the yaw arm connected with the yaw wheel and the yaw wheel cover are respectively provided with the oil storage grooves.

Further, one end of the ejector rod pull tab is connected with the bottom of the ejector rod and the other end is limited on the housing by a connecting rod rotating shaft.

Further, the long rotating shaft is further provided with fixing rings at both ends thereof, and the long rotating shaft is provided with a plurality of limiting holes for limiting the driven gear, the eccentric wheels and the yaw wheels, and the driven gear, the eccentric wheels and the yaw wheels are limited by limiting pins.

With the above structure, in operation of the shoulder belt massage machine core of the present disclosure, the battery supplies power to the circuit board, the circuit board controls the brushless motor to operate, and when the brushless motor rotates, the gear transmission group and the long rotating shaft connected with the gear transmission group are driven to rotate synchronously, and the long rotating shaft drives the eccentric wheel and the yaw wheel to rotate and thus drives the ejector rod to rotate, the ejector rod is limited by the ejector rod pull tab, so that the free end of the ejector rod moves up and down within a certain range. Because the yaw wheel and the yaw shaft have a yaw slope, the yaw arm arranged on the yaw shaft in a sleeving mode can swing left and right when the long rotating shaft is rotated by the guide block, the function of clamping the neck is realized, further, in combination with the ejector rod, it is possible to achieve the massaging effect of kneading. The present disclosure is driven by the brushless motor, and it is possible to simultaneously drive the yaw arm and the ejector rod to perform the right and left yaw movement and the up and down yaw movement to complete the kneading action. Compared with the existing double-drive structure, the overall structure of the disclosure is more compact, the manufacturing cost is lower, and the market competitiveness can be improved.

### BRIEF DESCRIPTION OF FIGURES

FIG. 1 is an exploded view of the structure of the present disclosure.

FIG. 2 is a combined schematic diagram of the present disclosure.

FIG. 3 is a cross-sectional view of the present disclosure.

FIG. 4 is a partial structural schematic diagram of the present disclosure.

FIG. 5 is a structural schematic diagram of an eccentric wheel of the present disclosure.

FIG. 6 is a structural schematic diagram of an ejector rod of the present disclosure.

FIG. 7 is a structural schematic diagram of the ejector rod in another direction of the present disclosure.

FIG. 8 is a structural schematic diagram of a yaw arm of the present disclosure.

FIG. 9 is a structural schematic diagram of the yaw arm in another direction of the present disclosure.

### DESCRIPTION OF REFERENCE NUMERALS

housing 1, a lower housing 1A, upper cover 1B, chamber 1C, guide groove 1D;
battery 2; brushless motor 3, worm 3A, fixing sleeve 3B; circuit board 4;
gear transmission group 5, helical gear 5A, spur gear 5B, driven gear 5C, gear rotating shaft 5D,
gear box 5E; long rotating shaft 6, limiting hole 6A; eccentric wheel 7;
yaw wheel 8, yaw shaft 8A, yaw slope 8B;
yaw wheel cover 9, ejector rod 10; yaw arm 11, yaw hole 11A,
guide block 11B, massage seat 11C, first massage seat 11D, second massage seat 11E;
massage assembly 12, first massage head 12A, duplex massage head 12B;
ejector rod pull tab 13, bearing 14, Hall plate 15, heating sheet 16, connecting rod rotating shaft 17, fixing ring 18.

### DETAILED DESCRIPTION

In order to further explain the technical solutions of the present disclosure, the present disclosure is described in detail below by specific embodiments.

In the description of the disclosure, it needs to be understood that, the terms “central”, “longitudinal”, “lateral”, “upper”, “lower”, “front”, “rear”, “left”, “right”, “vertical”, “horizontal”, “top”, “bottom”, “inner”, “outer” and the like indicate an orientation or positional relationship based on that shown in the drawings, only for ease of description of the disclosure and for simplicity of description, and are not intended to indicate or imply that the device or element referred to must have a particular orientation, be constructed and operate in a particular orientation, and therefore should not be construed as limiting the disclosure. In the description of the present disclosure, unless otherwise specified, the meaning of “plurality” is two or more.

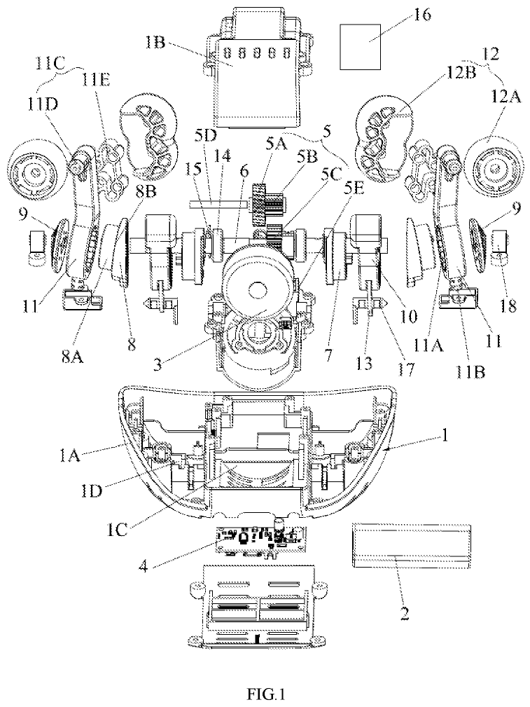

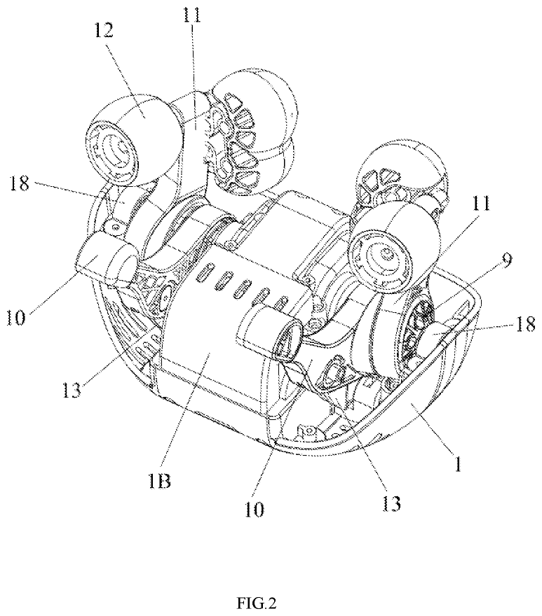

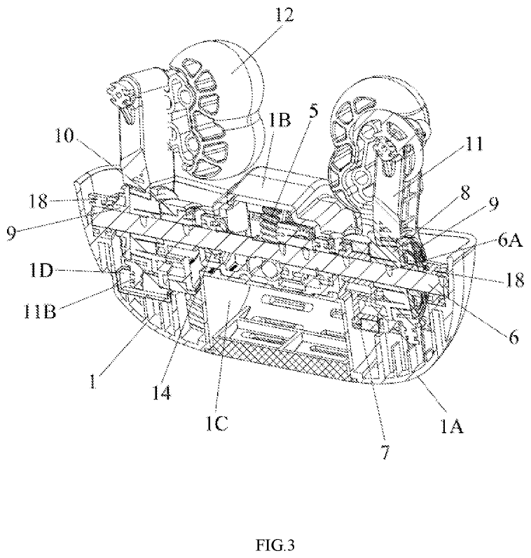

As shown in FIGS. 1 to 4, the present disclosure discloses a shoulder belt massage machine core, which includes a housing 1, a battery 2, a brushless motor 3, a circuit board 4, a gear transmission group 5, a long rotating shaft 6, eccentric wheels 7, yaw wheels 8, wheel covers 9, ejector rods 10, yaw arms 11 and massaging assemblies 12.

The battery 2, the brushless motor 3, the gear transmission group 5 and the long rotating shaft 6 are arranged inside the housing 1, the battery 2 is connected with the circuit board 4, the brushless motor 3 is connected with the circuit board 4, the gear transmission group 5 is connected with an output shaft of the brushless motor 3, the long rotating shaft 6 is connected with the gear transmission group 5, the two eccentric wheels 7 are fixedly arranged on both sides of the long rotating shaft 6 corresponding to the gear transmission group 5, the ejector rod 10 is arranged on the eccentric wheel 7 in a sleeving mode, one end of the ejector rod 10 connected with the eccentric wheel 7 is limited on the housing 1 by an ejector rod pull tab 13, the yaw wheels 8 are fixedly arranged outside the long rotating shaft 6 corresponding to the two eccentric wheels 7, a side of the yaw wheel 8 facing away from the eccentric wheel 7 is provided with a yaw shaft 8A, one end of the yaw arm 11 is provided with a yaw hole 11A corresponding to the yaw shaft 8A, the yaw hole 11A is arranged on the yaw shaft 8A in a sleeving mode, the yaw shaft 8A of the yaw wheel 8 and one end connected with the yaw arm 11 form a yaw slope 8B, the bottom of one end of the yaw arm 11 connected with the long rotation shaft 6 is provided with a guide block 11B, the guide block 11B is movably disposed in a guide groove 1D of the housing 1, the yaw hole 11A is disposed corresponding to the yaw slope 8B, the other end of the yaw arm 11 is provided with a massage seat 11C, the massage assembly 12 is mounted on the massage seat 11C, the yaw wheel cover 9 is arranged on the long rotating shaft 6 in a sleeving mode and fixed with an outer end surface of the yaw shaft 8A of the yaw wheel 8.

In operation of the shoulder belt massage machine core of the present disclosure, the battery 2 supplies power to the circuit board 4, the circuit board 4 controls the operation of the brushless motor 3, when the brushless motor 3 rotates, the gear transmission group and the long rotating shaft 6 connected with the gear transmission group 5 are driven to rotate synchronously, the long rotating shaft 6 drives the eccentric wheel 7 and the yaw wheel 8 to rotate and thus drives the ejector rod to rotate, the ejector rod is limited by the ejector rod pull tab 6, so that the free end of the ejector rod 10 moves up and down within a certain range. Because the yaw wheel 8 and the yaw shaft 8A have a yaw slope 8B, the yaw arm 11 arranged on the yaw shaft 8A in a sleeving mode can swing left and right when the long rotating shaft 6 is rotated by the guide block 11B, the function of clamping the neck is realized, further, in combination with the ejector rod 10, it is possible to achieve the massaging effect of kneading.

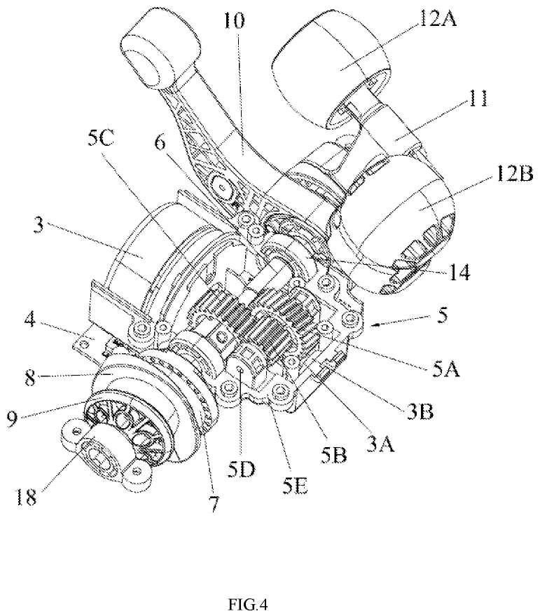

As shown in FIG. 4, In the present embodiment, the gear transmission group 5 includes a double gear having a helical gear 5A and a spur gear 5B and a driven gear 5C fixed on the long rotating shaft 6, the helical gear 5A and the spur gear 5B of the double gear are fixed together through a gear rotating shaft 5D, the gear transmission group 5 is mounted on a gear box 5E, the gear rotating shaft 5D is parallel with the long rotating shaft 6, two ends of the gear rotating shaft 5D are disposed on the gear box 5E; the brushless motor 3 is provided with a worm 3A, the worm 3A is perpendicular to the long rotating shaft 6 and is arranged below the long rotating shaft 6, one end of the worm 3A is connected with the brushless motor 3, the other end is sleeved with a fixing sleeve 3B, and the fixing sleeve 3B is fixed on the gear box 5E; the helical gear 5A of the double gear meshes with the worm 3A and the spur gear 5B meshes with the driven gear 5C. When the brushless motor 3 operates, the worm 3A rotates to decelerate the helical gear 5A and the spur gear 5B engaged with the worm 3A, and then the torque force is added to rotate the driven gear 5C and the long rotating shaft 6 fixed to the driven gear 5C. The gear box 5E is detachably mounted on the housing 1.

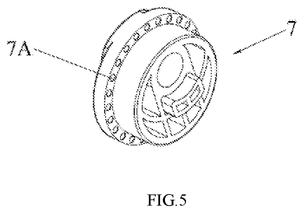

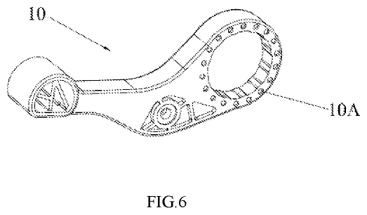

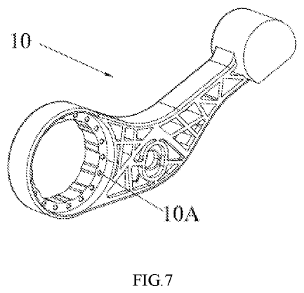

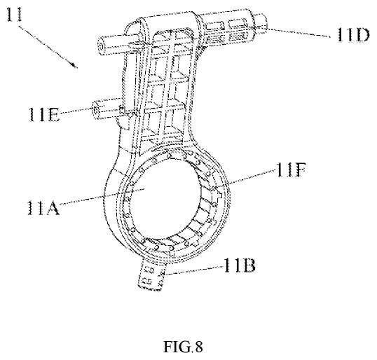

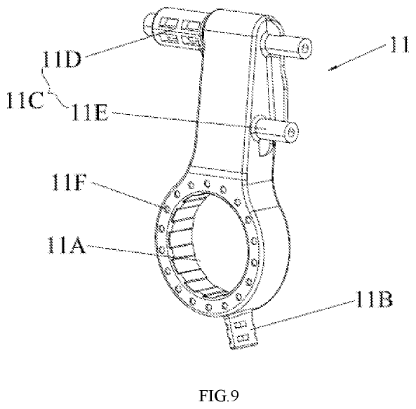

As shown in FIGS. 5 to 9, the eccentric wheel 7, the ejector rod 10 and the yaw arm 11 are respectively provided with a plurality of oil storage grooves 7A, 10A 11A, the oil storage grooves 7A of the eccentric wheel 7 are provided at one end surface of the eccentric wheel 7 connected with the ejector rod 10, two end surfaces of the ejector rod 10 connected with the eccentric wheel 7 and the yaw wheel 8 are respectively provided with the oil storage grooves 10A, and two end surfaces of the yaw arm 11 connected with the yaw wheel 8 and the yaw wheel cover 9 are respectively provided with the oil storage grooves 10A. When the oil storage grooves 7A, 10A, 11A are filled with the lubricant, oil film contact among the eccentric wheel 7, the yaw wheel 8, the yaw wheel cover 9, the ejector rod 10, and the yaw arm 11 can be made during the operation of the shoulder belt massage machine core of the present disclosure, thereby reducing frictional resistance and power loss, and also reducing wear between the lubricated components, and improving the service lives of the components.

In the present embodiment, the long rotating shaft 6 is further provided with bearings 14 on both sides of the driven gear 5C, the bearing 14 being provided between the eccentric wheel 7 and the driven gear 5C, wherein a Hall plate 15 is further provided between one eccentric wheel 7 and the bearing 14.

The shoulder belt massage machine core of the present disclosure is further provided with a heating sheet 16, the heating sheet 16 of the present embodiment is a heat-emitting film, the heat-emitting film is connected with the circuit board 4, a temperature control sensor is built in the heating sheet 16, and the heating sheet 16 is disposed on the inner top surface of the housing 1.

In the present embodiment, the housing 1 includes a lower housing 1A and an upper cover 1B, the center of the lower housing 1A is provided with a chamber 1C for mounting the gear box 5E and the brushless motor 3, The upper cover 1B covers the chamber 1C, and the heating sheet 16 is fixed in the upper cover 1B.

In the present embodiment, the massage assembly 12 includes a first massage head 12A disposed on the same side as the free end of the ejector rod 10 and a duplex massage head 12B disposed on the other side of the ejector rod 10, the massage seat 11C of the yaw arm 11 includes a first massage seat 11D extending from the yaw arm 11 to one side of a free end of the ejector rod 10 and a second massage seat 11E extending from the other side, the first massage head 12A is mounted on the first massage seat 11D, and the duplex massage head 12B is mounted on the second massage seat 11E In the present embodiment, the second massage seat 11E is an elastic block, and the duplex massage head 12B is mounted on the elastic block. During massage, the elastic block has deformation space in structure, which will deform when pressed and restore its shape when not stressed. The position of the massage head can be automatically adjusted according to the neck thicknesses of different people to improve the comfort of kneading massage. The first massage head 12A cooperates with an automatic end of the ejector rod 10 to perform the function of kneading.

In the present embodiment, one end of the ejector rod pull tab 13 is connected with the bottom of the ejector rod 10 and the other end is limited on the housing 1 by a connecting rod rotating shaft 17.

In the present embodiment, the long rotating shaft 6 is further provided with fixing rings 18 at both ends thereof, and the long rotating shaft 6 is provided with a plurality of limiting holes 6A for limiting the driven gear 5C, the eccentric wheels 7 and the yaw wheels 8, and the driven gear 5C, the eccentric wheels 7 and the yaw wheels 8 are limited by limiting pins.

The above embodiments and drawings are not intended to limit the form and pattern of a product of the disclosure, and any appropriate changes or modifications made thereto by those skilled in the art should be regarded as not departing from the patent scope of the pre sent disclosure.

What is claimed is:

1. A shoulder belt massage machine core, comprising: a housing (1), a battery (2), a brushless motor (3), a circuit board (4), a gear transmission group (5), a long rotating shaft (6), eccentric wheels (7), yaw wheels (8), yaw wheel covers (9), ejector rods (10), yaw arms (11) and massaging assemblies (12); the battery (2), the brushless motor (3), the gear transmission group (5) and the long rotating shaft (6) are arranged inside the housing (1), the battery (2) is connected with the circuit board (4), the brushless motor (3) is connected with the circuit board (4), the gear transmission group (5) is connected with an output shaft of the brushless motor (3), the long rotating shaft (6) is connected with the gear transmission group (5), the two eccentric wheels (7) are fixedly arranged on both sides of the long rotating shaft (6) corresponding to the gear transmission group (5), the ejector rod (10) is arranged on the eccentric wheel (7) in a sleeving mode, one end of the ejector rod (10) connected with the eccentric wheel (7) is limited on the housing (1) by an ejector rod pull tab (13), the yaw wheels (8) are fixedly arranged outside the long rotating shaft (6) corresponding to the two eccentric wheels (7), a side of the yaw wheel (8) facing away from the eccentric wheel (7) is provided with a yaw shaft (8A), one end of the yaw arm (11) is provided with a yaw hole (11A) corresponding to the yaw shaft (8A), the yaw hole (11A) is arranged on the yaw shaft (8A) in a sleeving mode, the yaw shaft (8A) of the yaw wheel (8) and one end connected with the yaw arm (11) form a yaw slope (8B), the bottom of one end of the yaw arm (11) connected with the long rotating shaft (6) is provided with a guide block (11B), the guide block (11B) is movably disposed in a guide groove (1D) of the housing (1), the yaw hole (11A) is disposed corresponding to the yaw slope (8B), the other end of the yaw arm (11) is provided with a massage seat (11C), the massage assembly (12) is mounted on the massage seat (11C), the yaw wheel cover (9) is arranged on the long rotating shaft (6) in a sleeving mode and fixed with an outer end surface of the yaw shaft (8A) of the yaw wheel (8).

2. The shoulder belt massage machine core according to claim 1, wherein, the gear transmission group (5) comprises a double gear having a helical gear (5A) and a spur gear (5B) and a driven gear (5C) fixed on the long rotating shaft (6), the helical gear (5A) and the spur gear (5B) of the double gear are fixed together through a gear rotating shaft (5D), the double gear is mounted on a gear box (5E), the gear rotating shaft (5D) is parallel with the long rotating shaft (6), two ends of the gear rotating shaft (5D) are disposed on the gear box (5E); the brushless motor (3) is provided with a worm (3A), the worm (3A) is perpendicular to the long rotating shaft (6) and is arranged below the long rotating shaft (6), one end of the worm (3A) is connected with the brushless motor (3), the other end is sleeved with a fixing sleeve (3B), and the fixing sleeve (3B) is fixed on the gear box (5E); the helical gear (5A) of the double gear meshes with the worm (3A) and the spur gear (5B) meshes with the driven gear (5C).

3. The shoulder belt massage machine core according to claim 1, wherein the gear transmission group (5) is mounted on a gear box (5E) which is detachably mounted on the housing (1).

4. The shoulder belt massage machine core according to claim 2, wherein the long rotating shaft (6) is further provided with bearings (14) on both sides of the driven gear (5C), the bearing (14) being provided between the eccentric wheel (7) and the driven gear (5C), wherein a Hall plate (15) is further provided between one eccentric wheel (7) and the bearing (14).

5. The shoulder belt massage machine core according to claim 3, further comprising a heating sheet (16) connected with the circuit board (4), the heating sheet (16) having a temperature control sensor built therein, and the heating sheet (16) being disposed on an inner top surface of the housing (1).

6. The shoulder belt massage machine core according to claim 5, wherein the housing (1) comprises a lower housing (1A) and an upper cover (1B), the center of the lower housing (1A) is provided with a chamber (1C) for mounting the gear box (5E) and the brushless motor (3), and the upper cover (1B) covers the chamber (1C), and the heating sheet (16) is fixed in the upper cover (1B).

7. The shoulder belt massage machine core according to claim 1, wherein the massage assembly (12) comprises a first massage head (12A) and a duplex massage head (12B), the massage seat (11C) of the yaw arm (11) comprises a first massage seat (11D) extending from the yaw arm (11) to one side of a free end of the ejector rod (10) and a second massage seat (11E) extending from the other side, the first massage head (12A) is mounted on the first massage seat (11D), and the duplex massage head (12B) is mounted on the second massage seat (11E); the second massage seat (11E) is an elastic block, and the duplex massage head (12B) has a mounting opening corresponding to the elastic block, the mounting opening is arranged on the elastic block in a sleeving mode.

8. The shoulder belt massage machine core according to claim 1, wherein the eccentric wheel, the ejector rod and the yaw arm are respectively provided with a plurality of oil storage grooves, the oil storage grooves of the eccentric wheel are provided at one end surface of the eccentric wheel connected with the ejector rod, two end surfaces of the ejector rod connected with the eccentric wheel and the yaw wheel are respectively provided with the oil storage grooves, and two end surfaces of the yaw arm connected with the yaw wheel and the yaw wheel cover are respectively provided with the oil storage grooves.

9. The shoulder belt massage machine core according to claim 1, wherein one end of the ejector rod pull tab (13) is connected with the bottom of the ejector rod (10) and the other end is limited on the housing (1) by a connecting rod rotating shaft (17).

10. The shoulder belt massage machine core according to claim 4, wherein the long rotating shaft (6) is further provided with fixing rings (18) at both ends thereof, and the long rotating shaft (6) is provided with a plurality of limiting holes (6A) for limiting the driven gear (5C), the eccentric wheels (7) and the yaw wheels (8), and the driven gear (5C), the eccentric wheels (7) and the yaw wheels (8) are limited by limiting pins.
